(19) United States
(12) Patent Application Publication
(10) Pub. No.: US 2009/0023629 A1
(43) Pub. Date: Jan. 22, 2009

(54) COMPOSITIONS COMPRISING POLYCATION-COMPLEXED PROTEIN CRYSTALS AND METHODS OF TREATMENT USING THEM

(75) Inventors: Susan Yun Qu, Auburndale, MA (US); Shubhang Mishra, Southborough, MA (US); Benjamin Paul Simeone, Wayland, MA (US); Sujit Kumar Basu, Cambridge, MA (US)

Correspondence Address: LOWRIE, LANDO & ANASTASI, LLP, ONE MAIN STREET, SUITE 1100, CAMBRIDGE, MA, 02142, US

(73) Assignee: ALTUS PHARMACEUTICALS INC., CAMBRIDGE, MA (US)

(21) Appl. No.: 12/158,384

(22) PCT Filed: Dec. 22, 2006
(86) PCT No.: PCT/US06/49278

§ 371 Date: Sep. 26, 2008

Related U.S. Application Data
(60) Provisional application No. 60/753,965, filed on Dec. 23, 2005.

(51) Int. Cl. A61K 38/00; A61P 1/00

(52) U.S. Cl. 514/2

(57) ABSTRACT

The present invention relates to compositions comprising polycation-complexed protein crystals and hyaluronic acid. Advantageously, the compositions of this invention are stable, long-acting and avoid local reactions at the injection site. Compositions according to this invention include sustained-release human growth hormone compositions. Such compositions are useful for treating a subject having a disorder associated with human growth hormone deficiency or that is ameliorated by human growth hormone therapy.

### TECHNICAL FIELD OF THE INVENTION

The present invention relates to compositions comprising polycation-complexed protein crystals and hyaluronic acid. Advantageously, the compositions of this invention are stable, long-acting and avoid local reactions at the injection site. Compositions according to this invention include, for example, sustained-release human growth hormone compositions. Such compositions are useful for treating a subject having a disorder associated with human growth hormone deficiency or that is ameliorated by human growth hormone therapy.

### BACKGROUND OF THE INVENTION

Proteins used in pharmaceuticals, for example, IFN-α, IFN-β, erythropoietin (“EPO”), human growth hormone (“hGH”), are delivered to patients by daily, or at least regular, subcutaneous, or other types of, injection. In order to alleviate the discomfort of repetitive injection, patients, especially children, would greatly benefit from a protein formulation that is long-acting, e.g., a once-per-week injection product, and non-irritating at the site of injection.

Current treatment regimens for hGH deficiency in humans are, for example, primarily based on hGH-delivery by subcutaneous injection. hGH plays a critical role in the regulation of cell and organ growth and in physiological function during various stages of development and aging. For example, overproduction of hGH results in gigantism in children and acromegaly in adults, whereas under-production leads to dwarfism in children [Mauras et al., J. Clin. Endocrinology and Metabolism, 85 (10) , 3653-3660 (2000); Frindik et al., Hormone Research, 51(1), 15-19 (1999); Leger et al., J. Clin. Endocrinology and Metabolism, 83(10), 3512-3516 (1998)], Turner's Syndrome (females only) [Bramswig, Endocrine, 15(1), 5-13 (2001); Pasquino et al., Hormone Research, 46(6), 269-272 (1996)] and chronic renal insufficiency [Carroll et al., Trends in Endocrinology and Metabolism, 11(6), 231-238 (2000); Ueland et al., J. Clin. Endocrinology and Metabolism, 87(6), 2760-2763 (2002); Simpson et al., Growth Hormone & IGF Research, 12, 1-33 (2002)]. In adults, hGH deficiency can affect metabolic processing of proteins, carbohydrates, lipids, minerals and connective tissue and can result in muscle, bone or skin atrophy [Mehls and Haas, Growth Hormone & IGF Research, Supplement B, S31-S37 (2000); Fine et al., J. Pediatrics, 136(3), 376-382 (2000); Motoyama et al., Clin. Exp. Nephrology, 2(2), 162-165 (1998)]. Other hGH deficiency-related disorders characterized by growth failure or problems include AIDS wasting syndrome [Hirschfeld, Hormone Research, 46, 215-221 (1996); Tritos et al., Am. J. Medicine, 105(1), 44-57 (1998); Mulligan et al., J. Parenteral and Enteral Nutrition, 23(6), S202-S209 (1999); Torres and Cadman, BioDrugs, 14(2), 83-91 (2000)] and Prader-Willi syndrome [Ritzen, Hormone Research, 56(5-6), 208 (2002); Eiholzer et al., Eur. L. Pediatrics, 157(5), 368-377 (1998)].

A number of products have been developed in an attempt to address the need for hGH therapeutics that are stable and long-acting and that, therefore, can be delivered by a less-frequent injection schedule. To that end, various drug delivery technologies, such as hydrogels [Katakam et al., J. Controlled Release, 49(1), 21-26 (1997); ); Kim and Park, J. Controlled Release, 80(1-3), 69-77 (2002)], liposomes [Weiner et al., J. Pharm. Sci., 74(9), 922-925 (1985)], oil emulsions [Yu et al., J. Pharm. Sci., 85(4), 396-401 (1996); [Zhao et al., J. Dairy Sci., 75 (11), 3122-3130 (1992)]] and biodegradable polymer microspheres [Jostel et al., Clin Endocrinol (Oxf), 62(5):623-627 (2005); Sun et al., J Pharmacol Exp Ther., 289(3) :1523-1532 (1999); Jones et al., Adv Drug Deliv Rev, 28(1) :71-84 (1997); Johnson et al., Nat Med, 2(7):795-799 (1996)], have been employed. However, the resulting hGH formulations often display an undesirable burst release of the drug or may be difficult to manufacture.

For example, Nutropin Depot®, is an injectable suspension of recombinant human growth hormone (rhGH)-containing polylactide-coglycolide (PLG) microspheres (see http://www.gene.com). Significant manufacturing costs have led to withdrawal of that product from the market (see http://www.bioworld.com/servlet/com.accumedia.web.Dispatcher?next=bioWorldHeadlines article&forceid=35754). Moreover, studies involving the administration of Nutropin Depot® in pediatric patients lead to adverse injection-site reactions, resulting in nodules, erythema, pain, bruising, itching, lipoatrophy and puffiness (see http://www.drugs.com/PDR/Nutropin Depot for Injectable Suspension.html).

Another product currently in development by LG Pharmaceuticals Inc. (South Korea), is a microparticle suspension formulation containing hGH, hyaluronate, lecithin, and triglyceride. Drawbacks of this product include tedious manufacturing steps, such as spray and vacuum drying during processing, and unfavorable delivery means, specifically, a delivery fluid that must be injected by means of a 26 gauge needle [Kim et al., J. Controlled Release, 104: 323-335 (2005); U.S. Application No. 2005/0100605; EP 0918535B1].

PCT patent applications WO 2004/060310 and WO 2004/060920, which are incorporated herein by reference in their entirety, refer to hGH formulations, including those comprising crystals of hGH complexed with polyelectrolytes (i.e., polycations). Such compositions are stable and capable of sustained hGH release for up to a period of 1 week. While the polycation-complexed hGH crystal components render these compositions stable and long-acting, there is the possibility that in some patients, the charged nature of the complexed crystal surface may lead to a local reaction, which includes mild redness and swelling at the injection site.

Hyaluronic acid (“HA”) is a naturally occurring glycosaminoglycan that is biocompatible and bioabsorbable [Mitchell et al., European Journal of Cardio-Thoracic Surgery, 8, 149-152 (1994)]. It forms a highly viscous fluid with exceptional lubricating properties and has been used in drug formulations [Wobig et al., Clin. Ther. 21(9), 1549-1562, 1999; Canoso et al., Ann. Rheum. Dis., 42(2), 171-175, April 1983]. To date, hyaluronic acid is uniformly mixed with a drug solubilized in solution and subjected to subsequent processing to form a solution, a gel, a solid particle or a matrix.

In addition, hyaluronic acid has been employed as a gel delivery system for sustained release of erythropoietin, as described in U.S. Pat. No. 5,416,071. Sustained release formulations have been also based on proteins encapsulated in crosslinked hyaluronic acid hydrogels [Yun et al., Biomaterials, 25(1), 147-157 (2003); PCT patent application WO2004/050712; Yui et al., J. Controlled Release, 25, 133-143 (1993); PCT patent application WO 2000/078357]. Alternatively, drugs have been encapsulated in hyaluronic acid microspheres to achieve sustained release [Kima et al., Journal of Controlled Release, 104, 323-335, 2005; Hahn et al., Pharmaceutical Research, 21 (8), August 2004; European patent 0918535 and PCT patent application WO1998/043664].

The clinical application and safety of hyaluronic acid has been established in a variety of other therapeutic areas. See, e.g., Friedman et al., Dermatol. Surg., 28(6), 491, June 2002; Duranti et al., Dermatol. Surg., 24(12), 1317-1325, December 1998; \dot{A}kermark et al., Clinical Drug Investigation, 22(3), 157-166, 1 March 2002; and Wobig et al., Clin. Ther., 20(3), 410-423, May-June 1998]. For example, Lalenti, Mediators of Inflammation, 3:4, 287-289, 1994, studied the effect of hyaluronic acid to reduce the edema caused by injection of saline solutions containing either poly-L-lysine or poly-L-lysine/HA into the paws of male Wistar rats.

### SUMMARY OF THE INVENTION

The present invention provides compositions comprising polycation-complexed protein crystals and hyaluronic acid. According to a preferred embodiment of this invention, the protein is human growth hormone or a human growth hormone derivative, the polycation is polyarginine and the composition is a sustained release, long acting composition. The preferred compositions according to this invention are useful in methods for treating a subject having a disorder associated with human growth hormone deficiency or ameliorated by treatment with human growth hormone.

The present invention provides methods for preparing compositions comprising polycation-complexed protein crystals and hyaluronic acid.

Other objects of the invention will be appreciated by those skilled in the art, in view of the disclosure herein.

### BRIEF DESCRIPTION OF THE DRAWINGS

FIG. 1 illustrates the percentage of total animal subjects showing a post-dose injection site reaction over time after administration of either a vehicle control or polyarginine-complexed hGH or HA polyarginine-complexed hGH- See Example 8.

FIG. 2 illustrates the average amount (mean±standard error) of hGH (ng/mL) present in five animal subjects per group over time after administration of either polyarginine-complexed hGH or HA polyarginine-complexed hGH. See Example 9.

FIG. 3 illustrates the percentage of total animal subjects showing a post-dose injection site reaction over time after administration of either a vehicle control or polyarginine-complexed hGH or β-glycerophosphate-polyarginine-complexed hGH. See Example 10.

### DETAILED DESCRIPTION OF THE INVENTION

The present invention is directed to compositions comprising polycation-complexed protein crystals and hyaluronic acid. The use of hyaluronic acid in the compositions of the present invention advantageously causes any potential local injection site reactions that might be caused by administration of polycation-complexed protein crystals, such as polycation-complexed hGH crystals, to be reduced or prevented.

More particularly, and without being bound by theory, it is believed that the negatively-charged hyaluronic acid acts as to reduce any potential excess positive charge of the polycation that is complexed to the protein crystal, thus rendering the crystal more compatible with that of the outer and inner skin surfaces of the subject undergoing treatment. This is believed to be the first use of hyaluronic acid to neutralize excess crystal charge for use in pharmaceuticals.

### Definitions

As used herein, a “polycation” refers to a polymer chain that has a net positive charge under an appropriate pH environment. Examples of polycations include, but are not limited to, polyarginine, polyhistidine, polylysine, polylysine-graft-imidazole acetic acid, protamine, histones, myelin basic protein, polymyxin B sulfate, bradykinin, spermine, putrescine, polyallyamine, linear poly(ethyleneimine), branched poly(ethyleneimine), DEAE-dextran, polyornithine, chitosan, modified derivatives of the above and mixtures thereof. In a preferred embodiment of this invention, the polycation is polyarginine.

“Co-crystallization” is defined as two different materials crystallizing into the same crystalline lattice. For example, a monovalent cation, divalent cation or polycation may crystallize into the same crystalline lattice as a protein having a negatively-charged side chains.

“Complexation” or “complex” refers to an interaction between two entities, as well as to the process enabling such an interaction. Complexation, for example, may be an electrostatic interaction between a negatively-charged side chain of a protein residue with a positively-charged group of either a monovalent cation, divalent cation or polycation. In a preferred embodiment of this invention, complexation refers to the electrostatic interaction between a negatively-charged amino acid side chain found on the surface of a protein crystal with a positively-charged group of a polycation (i.e., the polycation is adsorbed to the protein crystal via electrostatic interaction).

The term “polycation-complexed protein crystal” (e.g., polyarginine-complexed hGH crystal) according to this invention includes (1) protein crystals, whether prepared with or without cations such as monovalent or divalent cations, which crystals are complexed with a polycation and (2) proteins that are co-crystallized with polycations and with or without cations such as monovalent or divalent cations.

A “protein” crystal useful in the compositions of this invention typically carries a net negative surface. Examples of proteins carrying a negative charge include, but are not limited to, human growth hormone (“hGH”), erythropoietin (“EPO”), Etanercept (Enbrel), insulin, granulocyte colony stimulating factor (“GMCSF”), TNF-α, fibrolase, IL-1 P, recombinant tissue plasminogen activator, Orthoclone OKT3, Factor VIII, bovine somatotropin and interleukin 2. A protein crystal useful in the compositions of this invention may carry a net negative surface charge due to charged amino acid side chains, glycosylation or other chemical or genetic modifications of the molecule, or due to a modulation of complexation conditions such as pH or ionic strength.

A protein “crystal” refers to one form of the solid state of matter having a three-dimensional crystal lattice, which is distinct from the amorphous or semi-crystalline state. Crystals display characteristic features, including a lattice structure, characteristic shapes and optical properties, such as, e.g., birefringence. Determination as to whether a protein is in a crystalline state may be carried out by any method known in the art, e.g., X-ray diffraction or powder X-ray diffraction or transmission electron microscopy (TEM).

“Hyaluronic acid” (“HA”), which is also known in the art as “hyaluron” or “hyaluronan”, as used in this application includes hyaluronic acid and salts thereof, including “sodium hyaluronate”. Preferably, HA has a molecular weight of between 50 kD to 3000 kD, more preferably between 1000 kD to 2500 kD, and still more preferably between 1400 kD to 1800 kD, as measured by conventional techniques. According to a preferred embodiment of this invention, the HA is not purified from a human or animal source. This preferred HA may be produced by recombinant DNA techniques or by conventional fermentation and purification techniques using cells that produce the desired HA.

“Excipients” or “carriers” include amino acids, alcohols, carbohydrates, proteins, lipids, surfactants, polymers, polyamino acids, buffer substances, salts or electrolytes, and mixtures thereof. Although not an exhaustive list, excipients or carriers include: 1) amino acids, such as glycine, arginine, aspartic acid, glutamic acid, lysine, asparagine, glutamine and praline, as well as polyamino acids such as polyarginine, polylysine, etc.; 2) carbohydrates, e.g., monosaccharides such as glucose, fructose, galactose, mannose, arabinose, xylose, ribose; 3) disaccharides, such as lactose, trehalose, maltose, sucrose; 4) polysaccharides, such as maltodextrins, dextrans, starch, glycogen and hyaluronic acid; 5) alditols, such as mannitol, xylitol, lactitol, sorbitol; 6) glucuronic acid, galacturonic acid; 7) cyclodextrins, such as methyl cyclodextrin, hydroxypropyl-β-cyclodextrin and alike; 8) inorganic molecules, such as sodium chloride, potassium chloride, magnesium chloride, phosphates of sodium and potassium, boric acid, ammonium carbonate, ammonium phosphate, zinc slats, colloidal silica, magnesium, and trisilicate; 9) organic molecules, such as acetates, citrate, ascorbate, lactate; 10) emulsifying or solubilizing/stabilizing agents like acacia, diethanolamine, glyceryl monostearate, lecithin, monoethanolamine, oleic acid, oleyl alcohol, poloxamer, polysorbates, sodium lauryl sulfate, stearic acid, sorbitan monolaurate, sorbitan monostearate, and other sorbitan derivatives, polyoxyl derivatives, wax, polyoxyethylene derivatives, sorbitan derivatives; and 11) viscosity increasing reagents like agar, alginic acid and its salts, guar gum, pectin, polyvinyl alcohol, polyethylene oxide, cellulose and its derivatives, propylene carbonate, polyethylene glycol, hexylene glycol, tyloxapol. Salts of such compounds may also be used.

In a preferred embodiment of this invention, the excipient is selected from the group consisting of: Tris-HCl, polyethylene glycol, sodium acetate, polyarginine, hyaluronic acid and mixtures thereof.

“Growth Hormone” (“GH”) refers to a growth hormone that in nature is secreted by the pituitary gland in a mammal. Although not an exhaustive list, examples of such mammals include humans, monkeys, cows, horses and pigs. In a preferred embodiment of this invention, the mammal is a human. As used herein, GH can be produced in any conventional way. Preferably, it is produced recombinantly.

Human growth hormone (“hGH”) denotes a protein having an amino acid sequence, structure and functional characteristic of native human growth hormone. As used herein, human growth hormone also includes any isoform of native human growth hormone, including but not limited to, isoforms with molecular masses of 5, 17, 20, 22, 24, 36 and 45 kDa [Haro et al., J. Chromatography B, 720, 39-47 (1998)]. Thus, the term hGH includes the 191 amino acid sequence of native hGH, somatotropin, and the 192 amino acid sequence containing an N-terminal methionine (Met-hGH) and somatrem [U.S. Pat. Nos. 4,342,832 and 5,633,352)]. According to a preferred embodiment of this invention, the hGH (or any GH) is not purified from a human or animal source. It, however, may be derived from transfected cells in culture. The hGH (or any GH) may be produced by recombinant DNA techniques, by conventional peptide synthesis techniques, or combinations thereof. If produced by recombinant DNA technology, hGH is referred to as recombinant human growth hormone (“rhGH”). Met-hGH is typically prepared by recombinant DNA methodology. Examples of genes that encode different DNA sequences of hGH include hGH-N and hGH-V [Haro et al., J. Chromatography B, 720, 39-47 (1998); Bennani-Baiti et al., Genomics, 29, 647-652 (1995)]. hGH protein chains may be present in the GH as monomers, dimers and higher order structures.

A “human growth hormone derivative” refers to a protein having an amino acid sequence that is comparable to that of naturally occurring human growth hormone. The term “comparable” refers to an amino acid sequence that has at least 60%, preferably at least 80% and more preferably at least 90% homology to the 191 amino acid sequence of hGH. In various embodiments of the present invention, human growth hormone derivatives comprise substitution, deletion and insertion variants of hGH or Met-hGH, post-translationally modified hGH and Met-hGH proteins, including deamidation, phosphorylation, glycoslylation, acetylation, aggregation and enzymatic cleavage reactions [Haro et al., J. Chromatography B, 720, 39-47 (1998)], chemically modified hGH or Met-hGH proteins, polypeptide analogs and chemically synthesized peptides containing amino acid sequences comparable to those of hGH or Met-hGH.

The soluble form of hGH or an hGH derivative may be studied by a variety of methods, including reversed phase high performance liquid chromatography (RP-HPLC), size exclusion chromatography high performance liquid chromatography (SEC-HPLC) and hydrophobic interaction chromatography (HIC) [Wu et al., J. Chromatography, 500, 595-606 (1990); “Hormone Drugs”, FDA publication, (1982)]) On the other hand, the crystalline form of hGH or an hGH derivative may be studied by optical microscopy, X-ray diffraction or TEM. In general, the conditions of crystallization will determine the shape of a protein crystal, i.e., a shape selected from the group consisting of spheres, needles, rods, plates (hexagonals and squares), rhomboids, cubes, bipyramids and prisms.

A “cation crystal of human growth hormone or a human growth hormone derivative” refers to human growth hormone or a human growth hormone derivative that has been crystallized in the presence of a monovalent or divalent cation. The term “cation” refers to a positively charged atom or group of atoms.

The term “valency” is defined as an element's ability to combine with other elements, which is dictated by the number of electrons in the outermost shell of the atom and expressed as the number of atoms of hydrogen (or any other standard univalent element) capable of uniting with (or replacing) its atoms [Webster's New World Dictionary of Science, Lindley, D. and Moore T. H., Eds., Macmillan, New York, N.Y., 1998]. The term “monovalent cation” refers to ions carrying a positive charge that have a valence state of one and can be organic or inorganic in nature. Examples of monovalent inorganic cations include ammonium (NH₄⁺) and Group I elements of the periodic table (H⁺, Li⁺, Na⁺, K⁺, Rb⁺, Cs⁺, and Fr⁺).

Examples of organic monovalent cations include, but is not limited to, quaternary ammonium cations. Quaternary ammonium cations are positively charged polyatomic ions having the structure NR₄⁺ with R being alkyl groups.

A “therapeutically effective amount” refers to that amount of a composition that is sufficient to treat, prevent, reduce the severity, delay the onset, or reduce the occurrence of one or more symptoms of the illness or disease being treated.

“Formulating” or “formulation” refers to putting two or more ingredients together. For example, formulating a protein crystal of this invention includes mixing solution(s) with suspension(s), a suspension with other suspension(s), or solid phase ingredient(s) with solution(s) or suspension(s) under appropriate conditions such as temperature, pH and ionic strength.

The term “sustained-release composition” according to this invention relates to a composition wherein the pharmaceutical agent is active in a mammal for a duration longer than that when the pharmaceutical agent is administered not as part of the sustained-release composition.

The-term “activity” according to this invention relates to the effect of a pharmaceutical agent on a biological system, e.g., an animal or human body. Activity could be a biological activity, a pharmacodynamic effect or efficacy. For example, the activity of hGH of this invention may be measured, at different time points after a composition is administered into a mammal, by an elevation of serum insulin-like growth factor 1 (IGF-1) level, which results from serum GH concentration, or by the amount of body weight gain or tibial (bone) growth.

Compositions According to this Invention

According to this invention, compositions comprising polycation-complexed protein crystals and hyaluronic acid are advantageous in that they are stable, long-acting and have reduced local reactions at the injection site of subjects to whom they are administered.

According to this invention, polycation-complexed protein crystals are protein crystals, whether prepared with or without monovalent or divalent cations, that are complexed with a polycation. Alternately, polycation-complexed protein crystals are proteins co-crystallized with polycations, with or without monovalent or divalent cations.

The compositions of this invention may further comprise an excipient, preferably a pharmaceutically acceptable excipient. The compositions of this invention may be in any form suitable for injection, preferably in suspension form. More preferably, they are in a form suitable for injection using a needle having a gauge greater than or equal to 27, or greater than or equal to 30.

The molar ratio of protein crystal to polycation present in the compositions of this invention is preferably between about 1:250 to about 100:1, more preferably between about 1:250 to about 1:20, between about 1:20 to about 10:1, or between about 10:1 to about 100:1. In a more preferred embodiment, the molar ratio of protein crystal to polycation is between about 3:1 to 10:1. In a further preferred embodiment, the polycation is polyarginine.

Compositions comprising polycation-complexed hGH crystals and hyaluronic acid are characterized by an hGH concentration of greater than about 0.1 mg/mL. For example, the concentration of hGH may be between about 0.1 mg/mL and about 10 mg/mL. Alternatively, the compositions may be characterized by an hGH concentration between about 10 mg/mL and about 5O mg/mL or between about 50 mg/mL and about 100 mg/mL.

Preferably, when the polycation is polyarginine, the polyarginine concentration may be adjusted, so that it is sufficient to maintain an approximate 5:1 hGH:polyarginine (w/w) ratio and maintain low solubility and release of hGH of about 5 ng/mL. Preferably, when the polycation is protamine, the protamine concentration may be adjusted, so that it is sufficient to maintain an approximate 3:1 hGH:protamine (w/w) ratio and maintain low solubility and release of hGH of about 5 ng/mL. The final concentration of HA present in a composition of this invention will ultimately depend on the excess positive charge of the polycation complexed to the protein crystal surface, which in turn will depend on the amount of protein crystal and polycation included in the composition. The amount of HA present in the final formulation of this invention is preferably between about 0.01% to about 0.5% (w/v), most preferably between about 0.05% to about 0.2% (w/v).

Compositions comprising polycation-complexed hGH crystals and hyaluronic acid may also include the following components: mannitol—about 0.5 mg/mL to about 100 mg/mL; sodium acetate—about 5 mM to about 500 mM (preferably about 25 mM to about 150 mM; Tris HCl—about 5 mM to about 100 mM; pH about 6.0 to about 9.0 (preferably about 6.5 to about 8.5); PEG (MW 800-8000, preferably 3350, 4000, 6000 or 8000)—up to about 50% (w/v).

Alternatively, such compositions may optionally comprise: sucrose—up to about 100 mg/mL; amino acids (e.g., arginine and glycine)—up to about 50 mg/mL; preservatives (antimicrobial, phenol, metacrescol, benzyl alcohol, parabenzoate (paraben))—up to about 5% (preferably up to about 0.9%); and polysorbate—up to about 10 mg/mL.

A preferred embodiment of the present invention constitutes an injectable composition of polyarginine-complexed hGH crystals and hyaluronic acid. This composition comprises hGH crystals in a range of about 1 mg/mL to 200 mg/mL, preferably about 25 mg/mL, polyarginine in a range of about 0.1 mg/mL to about 100 mg/mL, preferably about 5 mg/mL, and a formulation vehicle. The formulation vehicle comprises (1) between about 10 mM and about 100 mM Tris-HCl, preferably about 25 mM Tris-HCl, at a pH range of between about 6 and about 9, preferably about pH 7.5, (2) between about 10 mM and about 500 mM NaOAc, preferably about 100 mM NaOAc, (3) between about 1% and about 50% (w/v) PEG-6000, preferably about 5% (w/v) PEG-6000, and (4) between about 0.01% and about 0.5% (w/v) hyaluronic acid, preferably about 0.2% (w/v) hyaluronic acid.

In another preferred embodiment of the present invention, polyarginine-hGH co-crystals are used. This co-crystal formulation composition comprises polyargine-hGH co-crystals with hGH in a range of about 1 mg/mL to 200 mg/mL, preferably about 25 mg/mL, polyarginine in a range of about 0.1 mg/mL to about 100 mg/mL, preferably about 5 mg/mL, and a formulation vehicle.

In a preferred embodiment, a formulation vehicle for compositions comprising hGH crystals and hyaluronic acid comprises about l00 mM sodium acetate, about 5% (w/v) PEG-6000 and about 25 mM Tris-HCl, pH 7.5, and 0.2% (w/v) HA. An hGH composition prepared using such a vehicle may comprise: about 25 mg/mL crystalline hGH and about 5 mg/mL polyarginine (or about 8.3 mg/mL protamine).

As will be appreciated by those of skill in the art, given that compositions according to this invention may comprise about 1 mg/mL to about 100 mg/mL hGH concentration, the polyarginine (or protamine) concentration should be adjusted, so that it is sufficient to maintain, in the more preferred embodiments of the invention, an approximate 5:1 hGH:polyarginine (w/w) ratio or an approximate 3:1 hGH:protamine (w/w) ratio and maintain low solubility and release of hGH of about 5 ng/mL. For example, for the above-described formulation, if the desired crystalline hGH concentration is about 20 mg/mL, the polyarginine concentration should be about 4 mg/mL (or protamine about 6.7 mg/mL).

The hGH containing compositions of this invention that are administered as a weekly injection display a relative bioavailability similar to that of daily injected soluble hGH in a mammal. The hGH included in the compositions of this invention has a relative bioavailability of at least 10% or greater compared to that of soluble hGH, delivered by the same route (e.g., subcutaneous or intramuscular injection), wherein said bioavailability is measured by the area under curve (AUC) of total in vivo hGH serum concentration for said soluble hGH and said crystal complex. Such hGH crystal complex is thus characterized by an advantageous in vivo release profile.

Methods for Producing Compositions According to this Invention

Illustrative methods for preparing polycation-complexed protein crystals are described in Examples 1-4 herein and in PCT patent applications WO 2004/060310 and WO 2004/060920, which are incorporated herein in their entirety by reference.

One embodiment of this invention relates to monovalent or divalent cation crystals of hGH or an hGH derivative complexed with a polycation, preferably polyarginine. According to this embodiment, crystallization of hGH is generally accomplished by preparing a buffered solution of hGH, purifying and/or desalting, dialyzing and concentrating the solution, and adding a cation to the solution. In a preferred embodiment, the cation is monovalent. In a more preferred embodiment, the monovalent cation is selected from the group consisting of: lithium, sodium, potassium and ammonium. In a most preferred embodiment, the monovalent cation is sodium. Addition of the monovalent cation results in the formation of a monovalent organic or inorganic cation bound to hGH or co-crystallized with it. The hGH starting material for crystallization is commercially available in lyophilized or frozen liquid form and is typically produced by recombinant DNA methods.

In this embodiment, the monovalent or divalent cation crystals of hGH referred to above are then complexed or co-crystallized with a polycation. For example, the monovalent or divalent cation crystals of hGH may be resuspended in a solution containing, inter alia, polycations such as protamine sulfate or polyarginine as shown in one of the above-preferred ratios or those exemplified in Examples 2-4.

In a preferred embodiment, crystals of hGH or an hGH derivative are crystallized with sodium acetate and subsequently either co-crystallized with or complexed with polyarginine or another polycation. The polyarginine typically has a molecular weight between about 1,500 and about 90,000 Daltons. Preferably, the crystals of hGH or an hGH derivative and polyarginine are present in an hGH:polyarginine ratio of about 5:1 to about 40:1 (w/w). That ratio may also range between about 10:1 to about 20:1 (w/w). Most preferably, that ratio ranges between about 3:1 to about 12:1 (w/w). According to an alternate embodiment, that ratio is about 5:1 to about 1:50 (w/w). In another embodiment, that ratio is between about 12:1 and about 15:1 (w/w). And, in a further embodiment, that ratio is about 5:1 (w/w).

The polycation crystals of hGH, prepared above, are then combined with hyaluronic acid. Upon isolation of the polycation crystals of hGH from solution, for example the ones described above (e.g., via centrifugation), the crystals are added to another solution comprising HA in the concentration range of about 0.01% to about 0.5% (w/v). That suspension is passed through a needle having a gauge size of between about 16 and about 32, and then incubated for more than 0.5 hour at a temperature of between about 2° C. and 8° C. for the electrostatic interaction to reach equilibrium. Preferably, the final concentration of HA in the suspension comprising these polycation crystals of hGH is preferably between about 0.01% to about 0.5%, most preferably between about 0.05% to about 0.2%.

One such method comprises the steps of: (a) forming a protein crystal; (b) complexing or co-crystallizing the protein crystal with a polycation to form a polycation-complexed protein crystal; and (c) formulating the polycation-complexed protein crystal with hyaluronic acid. In an alternate embodiment, the method comprises the steps of: (a) complexing the cation protein crystal with a polycation to form a polycation-complexed protein crystal; and (b) formulating the polycation-complexed cation protein crystal with hyaluronic acid. In a further embodiment, the method comprises the steps of: (a) co-crystallizing a protein or cation protein crystal with a polycation to form a co-crystal; and (b) formulating the co-crystal with hyaluronic acid. In a preferred embodiment of such methods, the protein is hGH and the polycation is polyarginine or protamine.

According to the methods of this invention, the addition of HA to the polycation complexed or co-crystallized hGH crystals results in various advantageous features. Some, but not all, of those features include: (1) the reduction of a mammal's injection site reaction after administration of polycation complexed or co-crystallized hGH crystals, (2) the ability to maintain a sustained release profile of polycation complexed or co-crystallized hGH crystals after administration in a mammal, (3) the ability to inject polycation complexed or co-crystallized hGH crystals into a mammal through a very fine gauged needle, e.g., 30-gauge needle, and (4) the preservation of crystallinity of hGH and integrity of complex in the polycation complexed or co-crystallized hGH crystals over time.

Use of Compositions According to this Invention

Methods for treating subjects using the compositions of this invention comprise the step of administering to a subject in need of said treatment a therapeutically effective amount of a composition comprising polycation-complexed protein crystals and hyaluronic acid. As used herein, the term “subject” includes mammals, including humans. According to a preferred embodiment, the methods of this invention comprise the step of administering to a subject a therapeutically effective amount of a composition comprising polycation-complexed hGH crystals and hyaluronic acid.

According to a further preferred embodiment, this invention relates to methods for improving injection site tolerance in a subject in need of protein therapy comprising the step of administering to the subject a composition comprising a polycation-complexed protein crystal and hyaluronic acid. In a further preferred embodiment, this invention relates to methods for improving injection site tolerance in a human undergoing human growth hormone therapy comprising the step of administering to the human a therapeutically effective amount of a composition comprising polycation-complexed protein crystals and hyaluronic acid.

Disorders related to hGH insufficiency that may be treated according to this invention include, but are not limited to: adult growth hormone deficiency, pediatric growth hormone deficiency, Prader-Willi syndrome, Turner syndrome, short bowel syndrome, chronic renal insufficiency, idiopathic short stature, dwarfism, hypopituitary dwarfism, bone regeneration, female infertility, intrauterine growth retardation, AIDS-related cachexia, Crohn's disease, Cystic Fibrosis, burns, as well as other genetic and metabolic disorders. In one embodiment of this invention, the disorder is pediatric growth hormone deficiency and treatment results in annualized growth velocity of between about 7 cm and about 11 cm in the child undergoing treatment.

The compositions of this invention may be administered by any conventional administration route including, for example, parenteral, oral, pulmonary, nasal, aural, anal, dermal, ocular, intravenous, intramuscular, intraarterial, intraperitoneal, mucosal, sublingual, subcutaneous, transdermal, topical, buccal or intracranial routes.

In a preferred embodiment, the compositions are administered by subcutaneous or intramuscular route.

In a further preferred embodiment, the compositions of this invention are administered by subcutaneous route, using a needle having a gauge greater than or equal to 27. Preferably, the needle gauge is greater than 30. The compositions may be administered from a pre-filled syringe or a meta dose infusion pump.

This invention advantageously permits sustained release of the protein component of the polycation-complexed protein crystal/hyaluronic acid compositions of this invention. In one embodiment of this invention, a polycation-complexed protein crystal/hyaluronic acid provides sustained release of hGH activity. The polycation-complexed hGH crystal/hyaluronic acid of this invention preferably provides sustained hGH activity for at least about 24 hours. More preferably, the polycation-complexed hGH crystal/hyaluronic acid of this invention provides sustained hGH activity for at least about 48 hours, 72 hours, 96 hours, 120 hours, or 144 hours. Even more preferably, the polycation-complexed hGH crystal/hyaluronic acid of this invention provides sustained hGH activity for at least about 1 week, 2 weeks, 3 weeks, or 4 weeks.

In one embodiment of this invention, sustained release compositions, including sustained release compositions comprising polycation-complexed hGH crystals and hyaluronic acid are administered about once every two days. In another embodiment, the compositions according to this invention are administered about once every three or four days. In yet another embodiment, the compositions according to this invention are administered about once a week. In another embodiment, the compositions according to this invention are administered about once every two weeks. In yet another embodiment, the compositions according to this invention are administered about once every month. It will be appreciated by those of skill in the art that the specific treatment regimen will depend upon factors such as the disease to be treated, the age and weight of the subject to be treated, general physical condition of the subject and judgment of the treating physician.

In a preferred embodiment of this invention, adults or children suffering from a variety of hGH insufficiencies, disease states or syndromes may be treated by various regimens of exogenously delivered hGH using compositions comprising polycation-complexed hGH crystals and hyaluronic acid according to this invention. For example, an endocrinologist may initiate therapy using a dose of about 0.2 mg/kg/week for a child, increasing the dose to about 0.3 mg/kg/week after several weeks or months of treatment, with the dose being further increased to about 0.7 mg/kg/week around puberty. As will be appreciated by those of skill in the art, the level of such exogenously delivered hGH dosed to adults or children requiring hGH delivery is also dependent upon the existing physiological level or concentrations of hGH.

Dosage regimens for hGH in adults or children are often expressed in terms of mg/kg or International Units (IU/kg). Such regimens are generally scheduled for either a day or-a week, i.e., mg/kg/day or mg/kg/week. Doses could also be fixed doses, primarily in adult growth hormone deficiency, i.e., mg/day or mg/week. With such considerations in mind, according to one embodiment of this invention, a single administration of a sustained-release composition comprising polycation-complexed hGH crystals and hyaluronic acid, for example, comprises a single weekly administration of about 9 mg hGH per 30 kg child, provides an in vivo hGH serum concentration of greater than about 10 ng/mL on days 1 and 2 post-administration, greater than about 5 ng/mL on days 3 and 4 post-administration and about 0.3 ng/mL on day 5 to day 7 post-administration.

Alternatively, a single administration of a sustained-release composition comprising polycation-complexed hGH crystals and hyaluronic acid, provides an in vivo hGH serum concentration of about 0.3 ng/mL to about 2,500 ng/mL hGH, preferably about 0.5 ng/mL to about 1,000 ng/mL hGH, most preferably about 1 ng/mL to about 100 ng/mL hGH for between about 0.5 hours and about 40 days post-administration in said mammal, preferably for between about 0.5 hours and any one of about 10 days, 7 days or 1 day post-administration. Similarly, a single administration of a composition comprising polycation-complexed hGH crystals and hyaluronic acid provides an in vivo serum concentration of above about 2 ng/mL hGH, preferably above about 5 ng/mL hGH, most preferably above about 10 ng/mL hGH for between about 0.5 hours to about 40 days post-administration in said mammal, preferably for any one of about 10, 7 or 1 day s post-administration. In a more preferred embodiment of this invention, a single administration of a composition comprising polycation-complexed hGH crystals and hyaluronic acid, provides an in vivo serum concentration of greater than about 0.3 ng/mL hGH for between about 0.5 hours and about 40 days in a mammal, preferably for any one period of any one of about 10, 7 or 1 day s post-administration.

According to one embodiment of this invention, a single weekly administration of a composition comprising polycation-complexed hGH crystals and hyaluronic acid, provides an in vivo hGH serum concentration of greater than about 10 ng/mL hGH on days 1 and 2 post-administration, greater than about 5 ng/mL hGH on days 3 and 4 post-administration and above about 0.3 ng/mL hGH on day 5 to day 7 post-administration. And, in a further embodiment, a single administration of a composition comprising polycation-complexed hGH crystals and hyaluronic acid, provides an in vivo serum concentration of greater than about 0.3 ng/mL hGH for between about 0.5 hours and about 10 days post-administration.

According to this invention, a single administration is defined as between about 0.01 mg/kg/week to about 100 mg/kg/week of a composition of this invention, for example, a composition comprising polycation-complexed hGH crystals and hyaluronic acid, wherein the volume of the administration is between 0.1 mL and about 1.5 mL. For example, pediatric growth hormone deficiency may be dosed with a composition comprising polycation-complexed hGH crystals and hyaluronic acid at about 0.3 mg/kg/week, e.g., about 9 mg hGH for a 30 kg child. Turner syndrome may be dosed with a composition comprising polycation-complexed hGH crystals and hyaluronic acid at about 0.375 mg/kg/week, e.g., about 11 mg hGH for a 30 kg child. Additionally, adult growth hormone deficiency may be dosed with a composition comprising polycation-complexed hGH crystals and hyaluronic acid at about 0.2 mg/kg/week, e.g., about 16 mg for a 80 kg adult, AIDS wasting disease may be dosed with a composition comprising polycation-complexed hGH crystals and hyaluronic acid at 6 mg/day, e.g., 42 mg/week.

In order that this invention may be better understood, the following examples are set forth. These examples are for the purpose of illustration only and are not to be construed as limiting the scope of the invention in any manner.

### EXAMPLES

As exemplified herein, hyaluronic acid improves the injection site tolerance and safety profile of polyarginine-complexed hGH in a rabbit acute tolerance model. In vivo results using hyaluronic acid polyarginine-complexed demonstrated a significant decrease in injection site reaction.

Because the high surface charge on polycation-complexed hGH may cause an injection site reaction in some subjects, a comparative study using a negatively charged small molecule glycerophosphate (GP) to bind to the positively charged polyarginine-complexed hGH crystalline surface was performed. Surprisingly, the HA-polyarginine-complexed hGH product resulted in a higher injection site tolerance than the GP-complexed counterpart. This demonstrated that it is not only the charge, but also the biocompatibility and polymeric nature, of hyaluronic acid, or a combination thereof, that render it useful to increase an injection site tolerance in subjects treated with polycation-complexed protein crystals.

### Example 1

### Crystallization of hGH with Sodium Acetate

Approximately 3.3 mL (10-20 mg/mL rhGH as supplied in phosphate buffer) of thawed rhGH feed solution was purified using a 10 DG-desalting column (BioRad) and concentrated using a concentrator (MWCO 10,000, Millipore). The concentration of hGH was in the range of 25 mg/mL, as measured by absorbance at 280 nm/0.75 (1 mg/mL hGH A₂₈₀=0.75 absorbance units). Crystals were grown by adding deionized water, and Tris-HCl (pH 8.6), PEG-6000 and Na-acetate to final concentrations of 100 mM, 6% (w/v) and 500 mM, respectively, in the total solution with a final protein concentration of 15 mg/mL. The solution was then mixed gently and incubated at 33° C. for 12-16 hours. Needle- or rod-like crystals with a length of 2 to 25 μm were obtained. The crystals can also be formed at temperatures between 33° C. and 15° C. but require increased crystallization time.

### Example 2

### Complexation of Sodium hGH Crystals with Cationic Polymer Additive

After crystallization yield was determined (see Example 1), sodium rhGH crystals were re-suspended in mother liquor (250 mM NaOAc, 100 mM Tris-HCl (pH 8.6), 6% PEG-6000, and either 7 mg/mL protamine sulfate or 4.2 mg/mL polyarginine) so that a final concentration of 21 mg/mL of sodium rhGH crystals was achieved. The protein to additive ratio for rhGH to protamine sulfate was approximately 3:1 (mg:mg) and for rhGH to polyarginine was 5:1 (mg:mg). These ratios are calculated to be mole ratios of approximately 1:1.7 for rhGH:protamine and approximately 1:0.6 for rhGH:polyarginine. The above rhGH pellets were uniformly re-suspended in isotonic mother liquor (without ionic polymer additive) comprising 100 mM NaOAc, 5% (w/v) PEG-6000 and 25 mM Tris-HCl pH 7.5 and stored at 4° C.

Additional rhGH:ionic polymer additive ratios may be obtained by varying the additive concentration (mg/mL) of the mother liquor while still resuspending to 21 mg/mL of rhGH. For example, increased concentrations of protamine sulfate (10.5 mg/mL) in the mother liquor can be used to obtain a ratio upon resuspension of rhGH:additive of 2:1.

### Example 3

### Co-Crystallization of hGH with Sodium Acetate and Protamine Sulfate

Approximately 3.3 mL (10-20 mg/mL rhGH as supplied in phosphate buffer) of thawed rhGH feed solution was purified using a 10 DG-desalting column (BioRad), and concentrated using a concentrator (MWCO 10,000, Millipore). The concentration of hGH was in the range of 25 mg/mL, as measured by absorbance at 280 nm/0.75 (1 mg/mL hGH A₂₈₀=0.75 absorbance units). Crystals were grown by adding deionized water, and Tris-HCl (pH 8.6), PEG-4000, protamine sulfate and Na-acetate to final concentrations of 100 mM, 6% (w/v), 2 mg/mL and 500 mM, respectively, in the total solution with a final protein concentration of 16 mg/mL. The solution was then mixed gently and incubated at 33° C. for 12-16 hours. Needle-like crystals were obtained ranging in length from approximately 2 to 25 μm.

### Example 4

### Co-Crystallization of hGH with Sodium Acetate and Polyarginine HCl

Approximately 3.3 mL (10-20 mg/mL rhGH as supplied in phosphate buffer) of thawed rhGH feed solution was purified using a 10 DG-desalting column (BioRad), and concentrated using a concentrator (MWCO 10,000, Millipore). The concentration of hGH was in the range of 25 mg/mL, as measured by absorbance at 280 nm/0.75 (1 mg/mL hGH A₂₈₀=0.75 absorbance units). Crystals were grown by adding deionized water, Tris-HCl (pH 8.6), PEG-4000, polyarginine HCl and Na-acetate to final concentrations of 100 mM, 2% (v/v), 2 mg/mL and 500 mM, respectively, in the total solution with a final protein concentration of 16 mg/mL. The solution was then mixed gently and incubated at 33° C. for 12-16 hours. Needle-like crystals were obtained ranging in length from approximately 2 to 25 μm.

### Example 5

### Preparation of HA-Polyarginine-Complexed hGH Crystals

Sodium hyaluronate was obtained from Novamatrix (Oslo, Norway) and had a molecular weight in the 1,400,000 to 1,800,000 g/mole range, calculated from intrinsic viscosity data. According to the manufacturer's product specification, the salt was fermented from Streptococcus zooepidemicus, harvested and purified to a very high degree of purity. As a result, the commercially-available hyaluronic acid salt may be used directly in vivo without the need for allergenic tests prior to administration.

In order to prepare HA-polyarginine-complexed hGH crystals, an appropriate amount of sodium hyaluronate was mixed in water (WFI) to achieve a 0.5% (w/v) stock solution of hyaluronic acid in water. The container was shaken vigorously and placed on a rotating rack for 4 to 6 hours to achieve a homogenous mixture. The solution was then filtered through a 0.2 μm membrane. Prior to use, this solution was stored at 5° C.

The polyarginine-complexed hGH crystals were centrifuged using a Beckman Centrifuge GS6R or Bench-top Centrifuge (Eppendorf centrifuge, model 5415D equivalent) at 3500 rpm for 10 minutes at 4° C. to separate the crystals from the mother liquor. The crystal pellet was resuspended in a final formulation vehicle (FV) consisting of 25 mM Tris, 100 mM NaOAc, 5% (w/v) PEG-6000, 0.01 to 0.3% (w/v) HA, and water (WFI) such that a final rhGH concentration of 25 mg/mL was achieved. The suspension was then passed through a 20 G needle to break up clumps and achieve a uniform suspension. This suspension was subsequently incubated at 2-8° C. overnight. The HA-polyarginine-complexed hGH crystals were then placed into vials or stored under refrigerated conditions.

### Example 6

### Preparation of β-Glycerophosphate(“GP”)-Polyarginine-Complexed hGH Crystals

In order to prepare GP-polyarginine-complexed hGH crystals, an appropriate amount of β-glycerophosphate disodium salt pentahydrate (MW 306, MP Biomedicals Catalog no. 195206) was mixed in water (WFI) to achieve a 20% (w/v) stock solution of GP in water. After dissolution, the mixture was filtered through a 0.2 μm membrane, and stored at 5° C.

The polyarginine-complexed hGH crystals were then centrifuged using Beckman Centrifuge GS6R or Bench-top Centrifuge (Eppendorf centrifuge, model 5415D equivalent) at 3500 rpm for 10 minutes at 4° C. to separate the crystals from the mother liquor. The crystal pellet was resuspended in a final formulation vehicle consisting of 25 mM Tris, 100 mM NaOAc, 5% (w/v) PEG-6000, 10% (w/v) GP, and water (WFI) such that a final rhGH concentration of 25 mg/mL was achieved. The suspension was then passed through a 20 G needle to break up clumps and achieve a uniform suspension. This suspension was subsequently incubated at 2-8° C. overnight. The GP-polyarginine-complexed hGH crystals was then placed into vials or stored under refrigerated conditions.

### Example 7

### Effect of HA on Polyarginine-Complexed hGH Crystals Dissolution Profile and Surface Charge

The dissolution rates and surface charges were studied in three samples: (1) polyarginine-complexed hGH crystals as a control, (2) HA-polyarginine-complexed hGH crystals (complexation using 0.01% HA), and (3) HA-polyarginine-complexed hGH crystals (complexation using 0.1% HA). The HA-polyarginine-complexed hGH samples were tested after residual, non-complexed HA was removed by centrifugation. Crystals were then resuspended in the original HA-free formulation vehicle (25 mM Tris-HCl, 100 mM NaOAc, 5% (w/v) PEG-6000, pH 7.5), as described in Example 5.

The dissolution rate was determined by Size Exclusion High Pressure Liquid Chromatography (HPLC) (Agilent Technologies). For example, polyarginine-complexed hGH crystals and HA-polyarginine-complexed hGH crystals were resuspended in either 25 mM Na-citrate (pH 2.5), 25 mM Na-citrate (pH 5.0), or 25 mM Na-citrate (pH 7.0), such that the final concentration of crystals in suspension was 2 mg/mL. The suspensions were then incubated at 37° C. for 15 minutes under constant stirring of 1000 rpm with the use of an Eppendorf Thermomixer. After incubation, an aliquot of the total suspension was obtained and diluted 1:1 with acidified dH₂O (pH 2.6). The remaining sample was then centrifuged to obtain a pellet and an identical volume taken of the total suspension was removed from the resulting supernatant and diluted 1:1 with acidified water (pH 2.6). Five microliter (μl) of each sample was then injected into the HPLC and the amount dissolved was determined by dividing the integrated hGH peak area resulting from the supernatant sample by the area of hGH in the total suspension sample. Integration included any and all degradation peaks.

Particle surface charges were measured using a Zetasizer Nano-Z (Malvern Instruments Ltd., UK). A Zeta Potential Transfer Standard was used to calibrate the instrument (Malvern catalog # DTS1050). For each sample, a 12 μL crystalline aliquot (concentration between 10 to 30 mg/mL) was added to 588 μL running buffer (20 mM Tris, pH 7.5). Thereafter, three measurements were taken at room temperature.

Results of the dissolution and surface charge experiments are reported in Table 1. These data demonstrated that at pH 2.5 and pH 7.0, the percent dissolution of the HA-polyarginine-complexed hGH samples was similar to that of the polyarginine-complexed hGH control. However, the HA-polyarginine-complexed hGH samples differed from the polyarginine-complexed hGH control at pH 5.0 in that the HA-complexed samples showed a reduced percent dissolution when compared to the control. The Zeta potential results illustrated a slight decrease of polyarginine-complexed hGH surface charge by the complexation of 0.01% HA, whereas a marked decrease in polyarginine-complexed hGH surface charge resulted when 0.1% HA was complexed to the crystal surface.

TABLE 1

| | % Dissolution | | | |
|---|---|---|---|---|
| Sample ID | pH 2.5 | pH 5.0 | pH 7.0 | ζ (mv) |
| polyarginine- | 101.68 | 70.93 | 13.39 | 26.00 |
| complexed hGH | | | | |
| crystals control | | | | |
| (batch 1) | | | | |
| polyarginine- | 97.35 | 55.47 | 13.79 | 24.53 |
| complexed hGH | | | | |
| crystals + 0.01% HA | | | | |
| polyarginine- | 99.52 | 56.51 | 12.14 | 17.81 |
| complexed hGH | | | | |
| crystals + 0.1% HA | | | | |

Additional concentrations of HA were used to formulate polyarginine-complexed hGH crystals and were tested for dissolution and surface charge. Results are shown in Table 2. At pH 5.0, the amount of dissolved polyarginine-complexed hGH crystals decreased with increased HA concentration. Moreover, the zeta potential results correlated with this trend, i.e., the more HA added, the lower the surface charge of the crystalline particles in this HA concentration range.

TABLE 2

% Dissolution

| Sample ID | PH 5.0 | pH 7.0 | ζ (mV) |
|---|---|---|---|
| polyarginine-complexed | 49.11 | 8.16 | 22.71 |
| hGH crystals control | | | |
| (batch 2) | | | |
| polyarginine-complexed | 36.39 | 7.38 | 15.59 |
| hGH crystals + 0.1% HA | | | |
| polyarginine-complexed | 29.16 | 7.04 | −3.07 |
| hGH crystals + 0.2% HA | | | |
| polyarginine-complexed | 22.49 | 6.44 | −25.23 |
| hGH crystals + 0.3% HA | | | |

### Example 8

### Effects of HA Complexation to Polyarginine-Complexed hGH Crystals on Animal Injection

Polyarginine-complexed hGH crystals were complexed with 0.2% HA following the protocol described in Example 5. The complexes were tested for dissolution rate and surface charge as shown in Example 7. The in vitro dissolution data demonstrated that these particular HA-polyarginine-complexed hGH crystals resulted in reduced dissolution of polyarginine-complexed hGH crystals at pH 5.0 and to a lesser extent at pH 7.0. Zeta potential data indicated a lower surface charge for the 0.2% HA-polyarginine-complexed hGH crystal samples than in the polyarginine-complexed hGH crystal control samples.

Rabbits (male New Zealand white, 8 and 16 per group, Charles River Labs) were shaved at the back (thoraco-lumbar area) and received a subcutaneous injection of 1 mL of a 25 mg/mL through a 30 G needle with one of the following samples:
(a) vehicle control (25 mM Tris-HCl, pH 7.5, 100 mM NaOAc, 5% (w/v) PEG-6000);
(b) polyarginine-complexed hGH crystals (25 mM Tris-HCl, pH 7.5, 100 mM NaOAc, 5% (w/v) PEG-6000, and 25 mg/mL crystallized rhGH complexed with 5 mg polyarginine); and
(c) 0.2% HA-polyarginine-complexed hGH crystals (25 mM Tris-HCl, pH 7.5, 100 mM NaOAc, 5% (w/v) PEG-6000, 0.2% (w/v) hyaluronic acid and 25 mg/mL crystallized rhGH complexed with 5 mg polyarginine. The injection sites were then inspected for swelling and/or redness over a period of seven days on a scale of 1-4. The sites were also inspected for ulceration, scratching and bleeding, but none of these symptoms were observed.

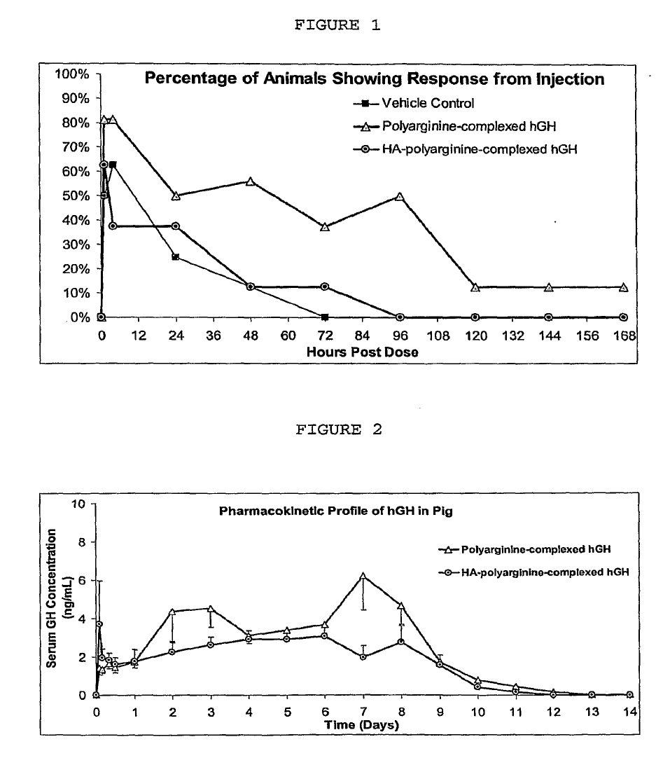

As shown in Tables 3 and 4 and FIG. 1, the percentage of animals showing injection site reaction was significantly lower in the HA-polyarginine-complexed hGH crystal group, as compared to the polyarginine-complexed hGH crystal control group. For example, at the 24-hour time point, the percentage of rabbits with injection site swelling in the 0.2% HA-polyarginine-complexed hGH crystal group was 12.5%, compared to 43.8% in the polyarginine-complexed hGH crystal group and 12.5% in vehicle control group. Throughout the observation period, the decrease in the percentage of animals showing swelling or redness over time was quicker in the 0.2% HA-polyarginine-complexed hGH crystal group over that of the polyarginine-complexed hGH crystal group. For example, at 96 hours (or day 4) after injection, the 0.2% HA-polyarginine-complexed hGH crystal group showed no signs of swelling or redness, whereas the polyarginine-complexed hGH crystal group continued to show an injection site reaction. The above observations demonstrate that HA formulation enjoys improved injection site tolerance both in terms of incidence of swelling and redness and speed at which swelling and redness was reduced.

TABLE 3
Percentage of Animals Showing Response¹ at Injection Site

| | Time Post Injection (hour) | | | | | | | | | |
|---|---|---|---|---|---|---|---|---|---|---|
| Sample ID | 0 | 1 | 4 | 24 | 48 | 72 | 96 | 120 | 144 | 168 |
| polyarg- | 0% | 81.25% | 81.25% | 50.00% | 56.25% | 37.50% | 50.00% | 12.50% | 12.50% | 12.50% |
| complexed | | | | | | | | | | |
| hGH | | | | | | | | | | |
| crystals | | | | | | | | | | |
| HA- | 0% | 62.50% | 37.50% | 37.50% | 12.50% | 12.50% | 0.00% | 0.00% | 0.00% | 0.00% |
| polyarg- | | | | | | | | | | |
| complexed | | | | | | | | | | |
| hGH | | | | | | | | | | |
| crystals | | | | | | | | | | |

¹Response is defined as % of animals that had either swelling, or redness or both.

TABLE 4
Percentage of Animals Showing Swelling at Injection Site

| | Time Post Injection (hour) | | | | | | | | | |
|---|---|---|---|---|---|---|---|---|---|---|
| Sample ID | 0 | 1 | 4 | 24 | 48 | 72 | 96 | 120 | 144 | 168 |
| Vehicle | 0.0% | 37.5% | 37.5% | 12.5% | 12.5% | 0.0% | 0.0% | 0.0% | 0.0% | 0.0% |
| Control² | | | | | | | | | | |
| polyarg- | 0.0% | 50.0% | 56.3% | 43.8% | 50.0% | 31.3% | 37.5% | 12.5% | 12.5% | 0.0% |
| complexed | | | | | | | | | | |
| hGH | | | | | | | | | | |
| crystals | | | | | | | | | | |
| HA- | 0.0% | 12.5% | 25.0% | 12.5% | 12.5% | 0.0% | 0.0% | 0.0% | 0.0% | 0.0% |
| polyarg- | | | | | | | | | | |
| complexed | | | | | | | | | | |
| hGH | | | | | | | | | | |
| crystals | | | | | | | | | | |

²Contained no polyarginine.

### Example 9

### Pharmacokinetics of HA-Polyarginine-Complexed hGH Crystals

A pharmacokinetics (PK) study using Yorkshire-Landrace pigs (Midwest Research, 5 pigs per group, male) was performed. Doses of 0.2% HA-polyarginine-complexed hGH crystals and polyarginine-complexed hGH Crystals were administered via subcutaneous injection to the back of the neck. At each time point after the injection, blood samples were collected from the anterior vena cava, and were tested for hGH concentrations.

Specifically, each group (n=5 each) received a single dose of either 0.2% HA-polyarginine-complexed hGH or polyarginine-complexed hGH crystalline formulation at 0.3 mg/kg. As shown in FIG. 2, the 0.2% HA-polyarginine-complexed hGH crystal group and the polyarginine-complexed hGH crystal group showed similar PK profiles (serum GH levels vs. time) and the length of hGH release was approximately the same for both groups. Consistent with the serum GH levels vs. time, similar pharmacodynamic results were observed in serum IGF-1 levels vs. time (data not shown) for HA-polyarginine-complexed hGH crystals and the polyarginine-complexed hGH crystals.

### Example 10

### Effects of GP-Polyarginine-Complexed hGH Crystals

Polyarginine-complexed hGH crystals were complexed with 10% GP as shown in Example 6. The complexes were tested for dissolution rate and surface charge as shown in Example 7. As illustrated in Table 5, GP-polyarginine-complexed hGH crystals achieved a low zeta potential of 5.45 mV and the in vitro dissolution profile was not significantly influenced by the GP complexation.

TABLE 5

| | |
|---|---|
| % Dissolution | ζ potential |

| Sample ID | PH 5.0 | pH 7.0 | (mV) |
|---|---|---|---|
| Polyarginine- | 99.30 | 36.13 | 23.61 |
| complexed hGH | | | |
| crystal control | | | |
| GP-polyarginine- | 93.29 | 32.29 | 5.45 |
| complexed. hGH | | | |
| crystals | | | |

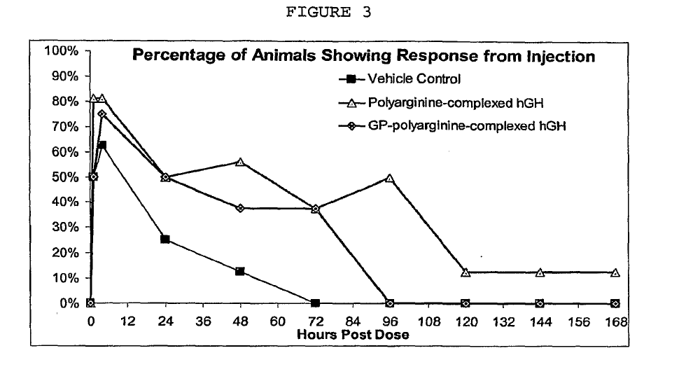

A rabbit study, described in Example 8, was performed. As shown in Tables 6 and 7 and FIG. 3, tolerance of GP-polyarginine-complexed hGH crystals at the injection site had not improved as significantly as that of 0.2% HA-polyarginine-complexed hGH crystals, even though the absolute value of zeta potential of GP-polyarginine-complexed hGH crystals was lower than that of 0.2% HA-polyarginine-complexed hGH crystals. This result signified that the ability of HA to greatly reduce the injection site reaction was not merely a result of charge neutralization. In fact, it may also due to the unique properties of HA. Thus, including HA in polyarginine-complexed hGH crystals enhances of safety profile without altering the hGH release profile.

TABLE 6
Percentage of Animals Showing Response (either swelling, or redness or both) at Injection Site

| | Time Post Injection (hour) | | | | | | | | | |
|---|---|---|---|---|---|---|---|---|---|---|
| Sample ID | 0 | 1 | 4 | 24 | 48 | 72 | 96 | 120 | 144 | 168 |
| polyarg- | 0.00 | 81.25% | 81.25% | 50.00% | 56.25% | 37.50% | 50.00% | 12.50% | 12.50% | 12.50% |
| complexed | | | | | | | | | | |
| hGH | | | | | | | | | | |
| crystals | | | | | | | | | | |
| GP- | 0.00 | 50.00% | 75.00% | 50.00% | 37.50% | 37.50% | 0.00% | 0.00% | 0.00% | 0.00% |
| polyarg- | | | | | | | | | | |
| complexed | | | | | | | | | | |
| hGH | | | | | | | | | | |
| crystals | | | | | | | | | | |

TABLE 7
Percentage of Animals Showing Swelling

| | | Incidence of Swelling between Day 2-8 | | | |
|---|---|---|---|---|---|
| | | (Percentage) | | | |
| Test | ξ potential | | 1 cm- | | Total |
| Articles | (mV) | <1 cm | 3 cm | >3 cm | (>1 cm) |
| Vehicle | NA | 0 | ⅛ (13) | 0 | ⅛ (13) |
| Control³ | | | | | |
| Polyarg- | +23.61 ± 1.08 | 1/16 (6) | 7/16 (44) | 1/16 (5) | 8/16 (50) |
| complexed | | | | | |
| hGH | | | | | |
| crystals | | | | | |
| GP-polyarg- | +5.43 ± 0.76 | 0 | ⅜ (38) | 0 | ⅜ (38) |
| complexed | | | | | |
| hGH | | | | | |
| crystals | | | | | |
| HA-polyarg- | −9.28 ± 0.58 | 0 | ⅛ (13) | 0 | ⅛ (13) |
| complexed | | | | | |
| hGH | | | | | |
| crystals | | | | | |

³Without polyarginine

### Example 11

### Efficacy of HA-Polyarginine-Complexed hGH Administered to Hypophysectomized Wistar Rats

An efficacy study in hypophysectomized male Wistar rats (Charles River, 9 rats per group, male) was performed. Doses of 0.2% HA-polyarginine-complexed hGH, or polyarginine-complexed hGH were administered via subcutaneous injection in the thoraco-lumbar region on either side of the spine. Test articles (0.2% HA-polyarginine-complexed hGH, and polyarginine-complexed hGH) were administered using 30 gauge, 8 mm needle attached to 0.3 mL syringe (BD part number 320438), which demonstrated that addition of HA does not impact the ease of administration of polyarginine-complexed hGH through fine gauge needles.

Specifically, each group (n=9 each) received a single dose of either 0.2% HA-polyarginine-complexed hGH, or polyarginine-complexed hGH crystalline hGH formulation at 5.6 mg/kg (0.56 mg/animal weighing about 0.1 kg). A control group (n=9) received seven daily doses of soluble hGH (Nutropin AQ) at the matched weekly dose (5.6 mg/kg) to crystalline hGH formulations. Body weights were measured weekly prior to the start of dosing and daily from Day-7 (seven days prior to dosing) until the end of the observation period on Day 15. For all the rats tested, the average weight on Day 1 (first day of test drug injection) is about 100 g.

The 0.2% HA-polyarginine-complexed hGH group and the polyarginine-complexed hGH group showed similar growth on Day 8 (one week growth): body weight of 115±6 g for 0.2% HA-polyarginine complexed hGH vs. 114±10 g for polyarginine complexed hGH. Moreover, daily growth hormone (Nutropin AQ) at the matched weekly dose of 5.6 mg/kg also produced similar growth (118±5 g). This result demonstrated that weekly injections of 0.2% HA-polyarginine-complexed hGH of the present invention had similar growth efficacy as weekly injections of polyarginine-complexed hGH crystals or daily hGH injections of soluble growth hormone.

### Example 12

### Integrity and Efficacy of HA-polyarginine Complexed hGH

A sample of 0.2% HA-polyarginine complexed hGH was produced as in Example 5. After preparation, the sample was stored at refrigerated (2-8° C.) condition for an extended period of time, for example, about 4 or more months. After storage, the sample was tested for its growth efficacy as in Example 11. The efficacy test showed that the stored sample of 0.2% HA-polyarginine complexed hGH afforded similar growth as a freshly prepared 0.2% HA-polyarginine complexed hGH on Day 8 (one week growth): body weight of 113±7 g vs. 115±6 g. This data demonstrated that the HA-polyarginine-complexed hGH did not lose crystallinity or the integrity of the complex in storage. Thus, the composition of the present invention maintained the integrity and efficacy of the polyarginine-complexed hGH and that hyaluronic acid as fabricated in the current invention did not disrupt, either during the manufacturing process, on long-term storage, or in use, the polyarginine-complexation of hGH crystals.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein, including the appended embodiments.

1. A composition comprising a polycation-complexed protein crystal and hyaluronic acid.

2. The composition according to claim 1, wherein said crystal is a cation crystal.

3. The composition according to claim 1, wherein said crystal is a monovalent cation crystal.

4. The composition according to claim 1, wherein the protein is a growth hormone or a growth hormone derivative.

5. The composition according to claim 4, wherein said growth hormone is human growth hormone.

6.-8. (canceled)

9. The composition according to claim 1, further comprising an excipient selected from the group consisting of: amino acids, salts, alcohols, carbohydrates, proteins, lipids, surfactants, polymers, polyamino acids and mixtures thereof.

10.-12. (canceled)

13. A method for treating a mammal in need of protein therapy comprising the step of administering to said mammal a therapeutically effective amount of a composition according to claim 1.

14. A method for treating a mammal having a disorder associated with human growth hormone deficiency, comprising the step of administering to said mammal a therapeutically effective amount of a composition according to claim 4.

15. The method according to claim 14, wherein said disorder is selected from the group consisting of: adult growth hormone deficiency, pediatric growth hormone deficiency, Prader-Willi syndrome, Turner syndrome, short bowel syndrome, chronic renal insufficiency, idiopathic short stature, dwarfism, hypopituitary dwarfism, bone regeneration, female infertility, intrauterine growth retardation, AIDS-related cachexia, Crohn's disease, Cystic Fibrosis and burns.

16.-19. (canceled)

20. A method for improving injection site tolerance in a mammal comprising the step of administering to said mammal a composition according to claim 1.

21.-22. (canceled)

23. A method for reducing the surface charge on a polycation-complexed protein crystal, comprising the step of adding hyaluronic acid to said polycation-complexed protein crystal.

24. A method for preparing a sustained release composition of a protein comprising the steps of:
(a) forming a protein crystal;
(b) complexing the protein crystal with a polycation to form a polycation-complexed protein crystal; and
(c) formulating the polycation-complexed protein crystal with hyaluronic acid.

25. A method for preparing a sustained release composition of a protein comprising the steps of:
(a) complexing a protein crystal with a polycation to form a polycation-complexed protein crystal; and
(b) formulating the polycation-complexed protein crystal with hyaluronic acid.

26. The method according to claim 24, wherein the protein crystal is a human growth hormone crystal.

27. The method according to claim 26, wherein the crystal is a monovalent cation crystal of human growth hormone.

28. The method according to claim 27, wherein the monovalent cation is sodium.

29. The composition according to claim 24, wherein said polycation is selected from the group consisting of polyarginine, polyhistidine, polylysine, polylysine-graft-imidazole acetic acid, protamine, histones, myelin basic protein, polymyxin B sulfate, bradykinin, spermine, putrescine, polyallyamine, linear poly (ethyleneimine), branched poly (ethyleneimine), DEAE-dextran, polyornithine, chitosan and mixtures thereof.

30. The method according to claim 29, wherein said polycation is polyarginine.

31. A method for preparing a sustained- release composition of a protein comprising the steps of:
(a) co-crystallizing a protein with a polycation to form a co-crystal; and
(b) formulating the co-crystal with hyaluronic acid.

32. A method for reducing or preventing an injection site reaction in a mammal caused by an administration of a polycation-complexed protein crystal comprising the step of formulating the polycation-complexed protein crystal with hyaluronic acid and injecting said formulation into said mammal.
